(19) United States
(12) Patent Application Publication
(10) Pub. No.: US 2025/0209402 A1
(43) Pub. Date: Jun. 26, 2025

(54) AGENTIC ARTIFICIAL INTELLIGENCE SYSTEM FOR AUTOMATED COMPLETION OF HEALTHCARE PROCESSES

(71) Applicant: OptraHEALTH Inc, San Jose, CA (US)

(72) Inventors: Abhijeet Sharadchandra Gholap, Los Gatos, CA (US); Gauri Gholap, Los Gatos, CA (US); Ashwin Kotwaliwale, Saratoga, CA (US); Nitin Sharma, Saratoga, CA (US)

(73) Assignee: OptraHEALTH Inc, San Jose, CA (US)

(21) Appl. No.: 19/077,031

(22) Filed: Mar. 11, 2025

Related U.S. Application Data
(63) Continuation-in-part of application No. 17/993,772, filed on Nov. 23, 2022.

(51) Int. Cl. G06Q 10/0633; G06F 40/279; G06F 40/30; G16H 10/60; G16H 40/20; G16H 80/00

(52) U.S. Cl. CPC G06Q 10/0633; G16H 10/60; G16H 40/20; G16H 80/00; G06F 40/279; G06F 40/30

(57) ABSTRACT

The present invention relates to agentic artificial intelligence (AI) systems for automating healthcare workflows, particularly in patient engagement and revenue cycle management (RCM). It can utilize natural language processing (NLP), machine learning, and recursive AI reasoning to interpret user instructions, extract insights from structured and unstructured data, and autonomously execute tasks within electronic medical records (EMR) and insurance systems. The system enables real-time patient interactions, adaptive workflow modifications, automated insurance verification, and discrepancy resolution, enhancing efficiency and accuracy in healthcare operations. And enables human/patient continuity of conversation for completing/resolving the underlying workflows.

### CROSS-REFERENCE TO RELATED APPLICATIONS

This application is a Continuation-in-Part of U.S. patent application Ser. No. 17/993,772, filed Nov. 23, 2022, the contents of which are incorporated herein by reference.

### TECHNICAL FIELD

The present disclosure relates generally to healthcare methods, and more specifically to methods and software systems for TeleHealth, health IT, digital health, counseling, etc.

### BACKGROUND

Health care spending in the U.S. increased to $4.1 trillion or $12,530 per capita in 2020. Accounting for 19.7 percent of the U.S. GDP and continuing to grow. Despite these high expenditures, the quality of American healthcare needs improvement. The U.S. consistently ranks below most developed countries when comparing factors such as access to care, care process, administrative efficiency, health care equity and health care outcomes.

The practice of medicine today relies heavily on analytical and diagnostic tools that are used to characterize a condition or disease. Often, the use of analytical and diagnostic tools involves determination of bioindicator levels to point to a symptom or disease. Moreover, the bioindicator data can suggest a general treatment plan because the advanced arts of pharmaceutical sciences and medicinal chemistry have identified drugs for many different conditions and diseases.

Drawbacks of the current practice of healthcare include inefficient use of the clinician's time in administering and monitoring a treatment plan. Other drawbacks include inefficient use of the clinician's time in gathering and analyzing bioindicator data to provide a diagnosis or prognosis. One way to improve healthcare is to provide for wellness. To deliver wellness to subjects, information can be provided to the subjects so that they can take control of their personal healthcare needs. For example, wellness information can include details concerning preventative medicine, use of pharmaceuticals, diet and nutrition, exercise, or self-abusive behavior.

Approaches to improve healthcare have included personalized health care. Conventional approaches to personalize treatment have typically separated people into different groups—with medical decisions, practices, interventions and/or products being tailored to the individual patient based on their predicted response or risk of disease. The terms personalized medicine, precision medicine, stratified medicine and medicine are used interchangeably to describe this concept, though some authors and organizations use these expressions separately to indicate particular nuances.

Software systems powered by computational linguistic algorithms are engaging users more meaningfully. Thus, enabling conversational information retrieval using interactive user interfaces (such as chatbots, voicebots, web and mobile apps, etc.). Such systems have gained popularity in healthcare where users are increasingly using such conversational tools.

A few examples where such systems are used are for educating users about medical topics, for an explanation of medical documents, for users to provide information to their providers, and payors, during TeleHealth, health IT, digital health, counseling, etc. Generally, conversational systems are coupled to a corpus from which information is retrieved and presented to the user, powered by semantic mapping, keyword search, etc., to understand the user's input. However, several scenarios are not efficiently handled by conversational systems. Scenarios such as, when a user's input is complex or when a sensitive medical topic is encountered, such scenarios should be answered by health experts. A few examples of such questions are “Am I going to get cancer?” or “I am having side effects; can I take another medication?” etc. Additionally, users will echo the same question in a variety of different ways. In such cases, the conversational system is unable to process the user's request further. This leaves the user confused and additionally the user's query is not answered. Several present day automated conversational systems function in this manner and even fewer conversational systems are trained to understand medical information.

The systems defined in the patent by Henry (US20190012390A1) refer to providing a live agent with more information about a conversation and the patent assigned to Conway (US1 0194029B2) is for analyzing data from online forums using computational linguistics. A patent granted to Nishant (U.S. Pat. No. 10,963,525B2) describes querying disconnected websites using conversational approaches and patent issues to Whitecotten in (U.S. Pat. No. 10,694,038B2) describe routing voice calls using context analysis in call centers. A patent was issued to Gholap et al (U.S. Pat. No. 10,754,882B2), that describes a method that converts any medical document into a knowledgegraph that is used to generate a medical scenario database cluster.

### SUMMARY OF INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims, drawings, and abstract as a whole.

The present invention relates to agentic artificial intelligence (AI) systems for automating healthcare workflows, particularly in patient engagement and revenue cycle management (RCM). It can use natural language processing (NLP), machine learning, and recursive AI reasoning to interpret user instructions, extract insights from structured and unstructured data, and autonomously execute tasks referencing healthcare information sources such as electronic medical records (EMR), LIMS and insurance systems. The system enables real-time patient interactions, adaptive workflow modifications, automated clinical and health insurance status, and discrepancy resolution, enhancing efficiency and accuracy in healthcare operations.

Embodiments of the invention include a method for machine-assisted healthcare workflow generation. The method can include steps of: (a) interpreting user instructions and extracting content using a natural language processor (NLP), (b) decomposing the extracted intent into logical workflows tasks with a workflow generation engine, (c) decomposing tasks into multi-step executable actions referencing healthcare systems, and (d) mapping tasks to predefined medical, administrative, and financial workflows, using a medical scenario classifier. In aspects, the method also includes a step of: (e) resolving discrepancies and routing conversations to an expert when deemed necessary (e.g., outside of a 95% confidence level) using a resolution manager.

In aspects, the method can include a step of building a medical database cluster to store medical scenarios that require input from a health expert. The medical database cluster can include, for example, data on signs/symptoms of medical conditions, data on severity of medical conditions, lab test results and social determinants of health and drugs and side-effects of drugs.

In aspects, a user submits user instructions by spoken natural language. In aspects, the step of interpreting user instructions and extracting content includes language analysis of the user's input using one or more of sentiment analysis and contextual identification of medical terminologies.

In aspects, the method also includes a step of retrieving and processing structured and unstructured medical data to optimize task execution. In aspects, the method also includes a step of verifying user credentials, insurance records, and/or patient eligibility for medical treatment or a medical procedure.

Embodiments also include a system for machine-assisted healthcare workflow generation. The system can include: (a) a natural language processing (NLP) module configured to interpret user instructions and extract intent, (b) a workflow generation engine configured to decompose the extracted intent into logical workflows tasks with recursion, and decompose tasks into multi-step executable actions referencing healthcare systems, (c) a medical scenario classifier configured to map tasks to predefined medical, administrative, and financial workflows, and (e) a resolution manager that can resolve discrepancies and route conversations to an expert when necessary (e.g., the system does not resolve a task within a set confidence level).

In aspects, the system also includes a medical database cluster. The medical database cluster can include, for example, data on signs/symptoms of medical conditions, data on severity of medical conditions, lab test results and social determinants of health and drugs and side-effects of drugs. The system of claim can also include a database of structured and unstructured medical data. The system can also include a database for verifying user credentials, insurance records, and/or eligibility for medical treatment or a medical procedure.

Embodiments include an agentic artificial intelligence (AI) system for healthcare automation. The system can include: (a) a natural language processing (NLP) module to interpret user instructions and extract intent, (b) a workflow generation engine that decomposes the intent into logical workflows tasks with recursion, (c) a workflow generation engine that decomposes tasks into multi-step executable actions referencing healthcare systems, (c) a medical scenario classifier that maps tasks to predefined medical, administrative, and financial workflows and (d) a resolution manager that can resolve discrepancies and route conversations to an expert when necessary.

In aspects, the AI agent dynamically interacts with users, including patients, healthcare providers, and administrative staff, to gather and/or verify information.

In aspects, the AI agent autonomously retrieves and processes structured and unstructured medical data, including physician notes, scanned documents, and historical patient records, to optimize task execution.

In aspects, the AI agent performs real-time insurance eligibility verification, validating coverage for one or more payers and detecting potential discrepancies.

In aspects, the AI agent identifies commonly executed medical procedures and tests and automatically initiates prior authorization requests when required.

In aspects, the AI agent performs real-time verification of user credentials, insurance records, and eligibility across multiple payors and systems, detecting discrepancies and ensuring compliance with required policies.

In aspects, the AI agent modifies workflows dynamically based on patient responses, risk factors, or newly available data, ensuring adaptive decision-making.

In aspects, the AI agent detects high-risk medical conditions and escalates the case to a healthcare professional for further review.

In aspects, the AI agent applies recursive resolution techniques to identify and resolve discrepancies before transferring the case to a human expert.

In aspects, the AI agent escalates tasks to human intervention only when AI-based resolution fails, ensuring efficient task automation with human oversight as needed.

In aspects, the AI agent maintains an audit trail of automated and human interventions to ensure compliance and traceability in healthcare workflows.

Embodiments also include method for machine-assisted automated continuation of conversations between a user, software system and healthcare professional, the method can includes steps of:
a) submitting user instructions to an agentic AI system,
b) validating if the user's inputs are medically relevant,
c) annotating the user's input for one or more of context, semantics, ontologies, medical terms and medical phrases,
d) mapping the inputs to the medical database cluster,
e) establishing a scenario assessment score based on combining information from mapping relevance, medical database cluster and ontologies, and
f) determining a score based on relevance (e.g., via confidence level), routing the conversation to a health expert or providing a response to the user and allowing the user to resume communication.

In aspects, the method also includes a step of building a medical database cluster to store medical scenarios that require input from a health expert. The medical database clusters can include, for example, data on signs/symptoms of medical conditions, data on severity of medical conditions, lab test results and social determinants of health and drugs and side-effects of drugs.

In aspects, the method also includes a step of of language analysis of the user's input using one or more of sentiment analysis and contextual identification of medical terminologies. In aspects, the method includes a step of sending context of the conversation to the health expert. In aspects, the method includes a step of storing anonymized conversations that from the user and health expert for model training. In aspects, the method includes a step of implementing a text-voice conversational system that is managed by a command, allowing the user to enter queries in natural language, and validating the user's queries for medical accuracy.

In aspects, the method also includes a step of
a) using a pre-processor to identify medical keywords in the user input,
b) extracting semantics and/or hidden relationships in the user input,
c) annotating user queries based on pre-trained data models for one or more of identifying/assigning, severity score, contextuality, intent and hidden medical terms/phrases.

In aspects, the method also includes a step of using a processor for mapping the user's input meta-data to pre-defined database cluster meta-data.

In aspects, the method also includes the steps of:
a) using a pre-processor to identify medical keywords in the user input,
b) using a processor to extract semantics and/or hidden relationships in the user input, and
c) annotating the user queries based on pre-trained data models for one or more of identifying/assigning, severity score, contextuality and intent.

In aspects, the method also includes steps of:
a) using a pre-processor to determine a scenario assessment score,
b) outputting the scenario assessment score,
c) continuing the automated conversation, and
d) directing the conversation with a health expert.

In aspects a processor generates medical scenario database clusters by analyzing health records, literature and/or medical information. In aspects, a processor sends the context of the user's conversation to a health expert before initiating contact with the health expert. In aspects, a processor stores user-health expert interactions, learns from the user-health expert interactions and re-annotates scenario database clusters. In aspects, the response to the user is a recommended treatment plan.

Embodiments also include a system for automating healthcare workflow and providing a treatment recommendation. The system can include:
a) a graphical user interface,
b) a server; and
c) software comprising a deep learning module configured to generate a workflow;
   The system can be configured to validate input from a user to determine medical relevance, annotate the user's input for one or more of context, semantics, ontologies, medical terms and medical phrases, map the inputs to a medical database cluster, establish a scenario assessment score based on combining information from mapping relevance, medical database cluster and ontologies and route the conversation to a health expert or providing a response to the user.

### BRIEF DESCRIPTION OF THE DRAWINGS

The accompanying drawings illustrate aspects of the present invention. In such drawings:

FIG. 1 is a block diagram that shows steps of a method that provides the automated continuation of the conversation between a user and health experts.

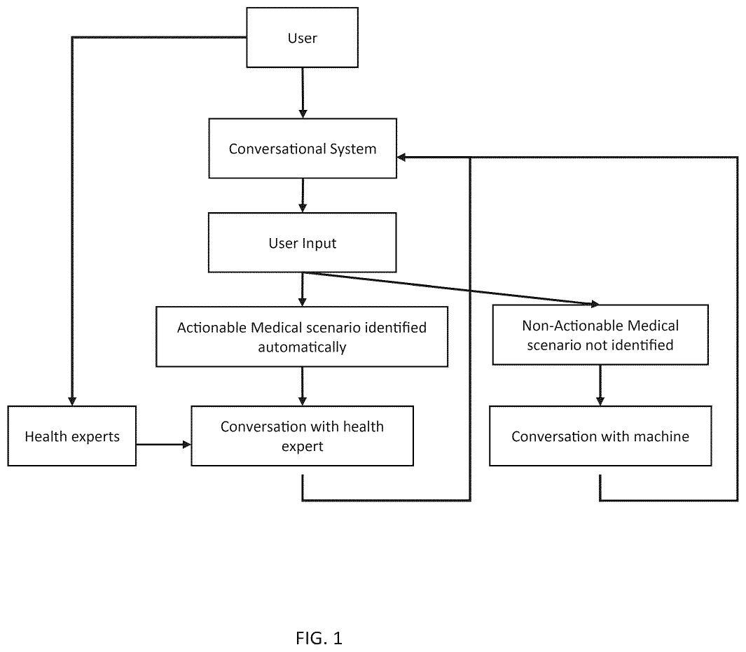

FIG. 2 is a block diagram that shows processors used in determining conversation meta-data to further determine criticality.

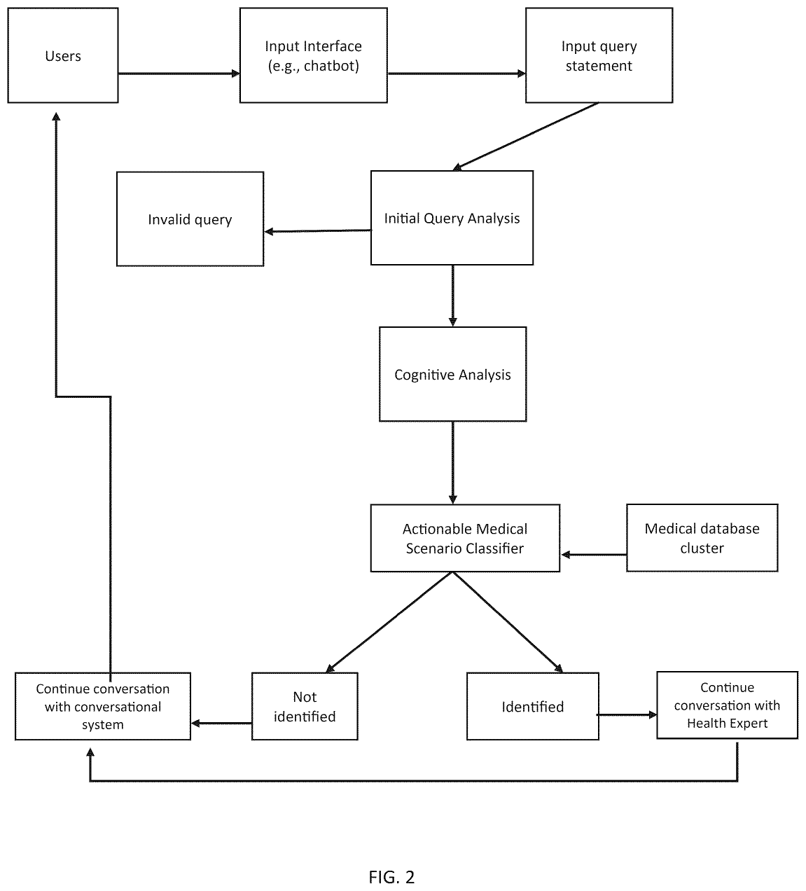

FIG. 3 is a block diagram that displays the process for generating medical database clusters used for storing specific medical scenarios that require conversation redirection and continuity with health experts

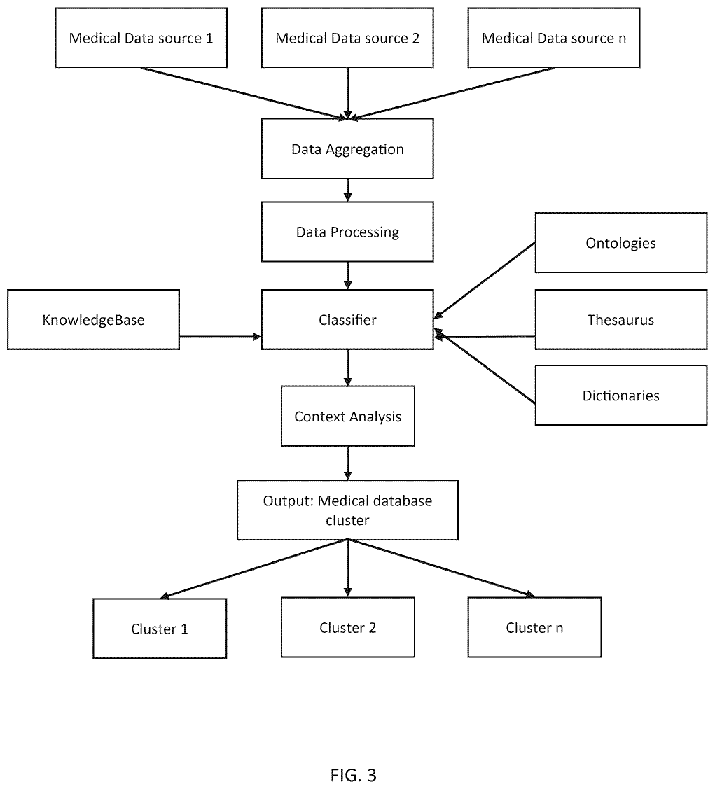

FIG. 4 is a flowchart that shows steps in a method of using an AI agent to produce a healthcare process.

FIG. 5 is a flowchart that shows steps in a method of using an AI agent to communicate with a user (e.g., patient).

FIG. 6 is a flowchart that shows steps in a method of using an AI agent to communicate with a user (e.g., healthcare professional) to develop a healthcare process.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

The term “computer learning” or “machine learning” refers to an application of artificial intelligence (AI) that provides systems the ability to automatically learn and improve from experience without being explicitly programmed. Machine learning focuses on the development of computer programs that can access data and use it to learn for themselves.

The term “artificial Intelligence” or “AI” refers to intelligence exhibited by machines, rather than humans. The term, as applied herein, refers to when a machine mimics “cognitive” functions that humans associate with other human minds, such as “learning” and “problem solving.”

The term “machine learning software” generally refers to a type of software application that uses artificial intelligence to make predictions or decisions based on data.

The term “deep learning” generally refers to a type of machine learning based on artificial neural networks in which multiple layers of processing are used to extract progressively higher-level features from data.

The term “module” such as that used in “deep learning module” generally refers to an extension to a software application's main program that is dedicated to a specific function. For example, a “deep learning module” refers to an extension dedicated to deep learning which can perform all deep learning functions automatically without additional programming.

The term “neural network” refers to a method in artificial intelligence that teaches computers to process data in a way that is inspired by the human brain. It is a type of machine learning process, called deep learning, that uses interconnected nodes or neurons in a layered structure that resembles the human brain.

“Artificial neural networks” or “ANNs” are distributed computing systems that include a number of neurons interconnected through connection points called synapses. Each synapse encodes the strength of the connection between the output of one neuron and the input of another. The output of each neuron is determined by the aggregate input received from other neurons that are connected to it. Thus, the output of a given neuron is based on the outputs of connected neurons from preceding layers and the strength of the connections as determined by the synaptic weights. An ANN is trained to solve a specific problem (e.g., pattern recognition) by adjusting the weights of the synapses such that a particular class of inputs produce a desired output.

The term “big data” refers to large data sets that can be analyzed computationally to reveal patterns, trends, and associations, especially relating to human physiological responses, human behavior and interactions. This can include, for example, a databases of electronic health records from a hospital or clinic.

The term “classifier” refers to the mathematical function, implemented by a classification algorithm that maps input data to a category. An algorithm that implements classification, especially in a concrete implementation, is known as a classifier. Similarly, “classification” refers to the process of recognition, understanding, and grouping of objects and ideas into preset categories (“sub-populations”). With the help of these pre-categorized training datasets, classification in machine learning programs leverage a wide range of algorithms to classify future datasets into respective and relevant categories. In aspects, the methods described herein utilize a medical scenario classifier to, for example, map tasks to predefined medical, administrative and financial workflows.

The term “historical data” refers to data (e.g., physiological measurements and actions of individuals) recorded and/or stored in a database that can be accessed for analysis and/or comparison. Historical data can include (a) data compiled from groups/populations of individuals and (b) data compiled from an individual person related to an ailment (e.g., kidney disease) and related treatments/complications. For example, data can be recorded from healthy people and people with known ailments. An analysis of the data can indicate variations in physiological measurements that can be correlated with ailments. Likewise, data from an individual person can be recorded and stored. This can allow the system to identify patterns, variations and/or aberrations in activity for that particular person.

The term “software as a service” or “SaaS” refers to a software licensing and delivery model in which software is licensed on a subscription basis and is centrally hosted. SaaS is also known as on-demand software, web-based software, or web-hosted software. SaaS is considered to be part of cloud computing, along with several other as a service business models. SaaS apps are typically accessed by users of a web browser (a thin client). SaaS became a common delivery model for many business applications, including office software, messaging software, development software, gamification, virtualization, etc. In aspects, the systems described herein are offered as SaaS.

The term “decompose” in computer science refers to the process of breaking down complex problems into smaller, more manageable parts. It is considered a fundamental problem-solving technique and one of the cornerstones of computer science. Different types of decomposition are defined in computer sciences. In structured programming, algorithmic decomposition breaks a process down into well-defined steps. Structured analysis breaks down a software system from the system context level to system functions and data entities. Object-oriented decomposition breaks a large system down into progressively smaller classes or objects that are responsible for part of the problem domain.

The term “corpus” refers to a collection of texts, such as medical records, used for research or analysis. It can also include medical histories, financial information, insurance information, etc.

The term “clustering data” refers to the process of partitioning a set of data objects into subsets. Each subset is a cluster, such that objects in a cluster are similar to one another, yet dissimilar to objects in other clusters. Accordingly, “clustered data” occurs when data points are not independent, but rather grouped into clusters or units, such as patients within hospitals, families within communities, or repeated measurements on the same individuals over time. Biomedical researchers often encounter clustered data, especially in fields like epidemiology and public health or in clinical trials. Analyzing clustered data properly is crucial to account for the non-independence of observations and obtain accurate and valid results. In aspects, the methods described herein utilize clustered data in, for example, constructing workflows.

The term “decomposition” or “factoring” in computer science refers to breaking a complex problem or system into parts that are easier to conceive, understand, program, and maintain. In aspects, the methods described herein utilize decomposition in, for example, constructing workflows.

As used herein, “additional biomedical information” refers to one or more evaluations of an individual that are associated with their health condition. Accordingly, “additional biomedical information” includes any of the following: physical descriptors of an individual, the height, weight and/or BMI of an individual, the gender of an individual, the ethnicity of an individual, family history, smoking history, occupational history, etc. Additional biomedical information can be obtained from an individual using routine techniques known in the art, such as from the individual themselves by use of a routine patient questionnaire or health history questionnaire, etc., or from a medical practitioner, etc. In aspects, the methods described herein utilize additional biomedical information in, for example, constructing workflows.

The term “prognosis” refers to the likely outcome or course of a disease and/or the chance of recovery or recurrence (e.g., a kidney disease). This is in contrast to a “diagnosis” which refers to identifying an ailment or disease, usually from examining a subject.

The term “health evaluation” or “health assessment” refers to a plan of care that identifies the specific needs of a person and how those needs will be addressed by the healthcare system or healthcare provider. Conventionally, a health assessment follows an evaluation of a subject's health status by performing a physical exam after taking a health history.

The term “workflow” refers to the sequence of industrial, administrative, or other processes through which a project passes from initiation to completion. Clinical workflow refers to a process involving a series of tasks performed by various people within and between work environments to deliver healthcare. Accomplishing each task may require actions by one person, between people, or across organizations—and can occur sequentially or simultaneously. Similarly, a medical workflow is a system that ensures patients are treated consistently and thoroughly. It includes a series of activities, such as check-ins, diagnostic tests, and treatments, that healthcare providers perform.

The term “health information exchange workflow” or “HIE workflow” refers to the workflow associated with Health Information Exchange (i.e., the electronic sharing of healthcare-related information).

The term “confidence level” or “CL” refers generally to a statistical measure of the percentage of test results that can be expected to be within a specified range. For example, a Confidence Level of 95% means that the result of an action will probably meet expectations 95% of the time.

The term “structured data” refers to data that is organized information that follows a predefined format (e.g., fitting into rows and columns such as a database table). In contrast, “unstructured data” is raw, unorganized information that does not conform to a set structure (e.g., images, audio, and large text documents where extracting specific data is more complex). Essentially, structured data is easily searchable and analyzed with traditional methods, whereas unstructured data requires advanced techniques to extract meaningful insights.

The term “natural language processing” or “NLP” refers to a field of artificial intelligence that allows computers to understand, interpret, and manipulate human language, enabling them to process and generate text or speech in a way that mimics human communication. NLP can be used in various applications like chatbots, email filtering, search engines, machine translation, sentiment analysis, text summarization and virtual assistants.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pain” refers to a distressing feeling often caused by intense or damaging stimuli. The International Association for the Study of Pain defines pain as “an unpleasant sensory and emotional experience associated with, or resembling that associated with, actual or potential tissue damage.” The terms “pain” and “discomfort” can be used interchangeably. The visual analogue scale (VAS) and numeric rating scale (NRS) are most commonly used to assess the present intensity of acute pain.

The term “physiological event” refers to a response or reaction of the body to a stimulus. Most are automatic/instinctive physiological responses. The healthy state of the body depends upon the integrity of various organ systems. The organ systems in the body function in a particular manner constantly. The mechanisms, by which the organ systems of the body function, can be referred to as “physiological mechanisms.” Physiological mechanisms explain any health-related events or outcomes. Physiological mechanisms can be altered voluntarily. For example, exercise causes alteration in the cardiac physiology of resting state.

The term “comorbid disorders” refers to additional diseases or conditions that a subject has at the same time as a primary health concern. Conditions described as comorbidities are often chronic or long-term conditions. In aspects, comorbidities are considered in the methods described herein (e.g. in constructing workflows).

The term “environmental factor” refers to exposures to substances (e.g., pesticides, industrial waste, etc.) where a subject lives or works. Common environmental factors include (a) chemicals (e.g., mold, pesticides, etc.), (b) air pollution, (c) climate change and natural disasters, (d) diseases caused by microbes, (e) lack of access to health care, (f) infrastructure issues, (g) poor water quality and (h) global environmental issues. In aspects, environmental factors are considered in the methods described herein.

The term “revenue cycle management” or “RCM” refers to a financial process that helps healthcare providers bill patients, track payments, and collect money. Steps in the RCM process can include, for example:
a) patient registration (register patients and verify their insurance),
b) eligibility verifications (confirm insurance coverage before services are rendered),
c) provide cost estimations (including co-pay when applicable),
d) prior-authorization (common when insurance companies require healthcare provider to get pre-approval for a treatment or procedure),
e) coding and charge capture (transcribe medical services into billable charges using medical codes),
f) claim submission (submit claims to insurance companies),
g) claim status inquiry (check the status of claims),
h) denial management (review and appeal denied claims),
i) payment posting (post payments to patient accounts), and
j) patient statements (create patient statements).

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the methods and systems particularly pointed out in the written description and claims hereof.

### AI Agent for Healthcare Processes

Managing healthcare processes requires significant administrative effort, often involving manual intervention that can lead to inefficiencies, delays, and errors. Existing automated solutions, such as virtual assistants, chatbots, and voicebots, provide partial automation but lack the ability to determine when human expertise is required, particularly in complex medical or financial interactions.

The field of computational linguistics has evolved significantly in the past decade. Computer software-based systems are leveraging this advancement, especially in healthcare. Such systems use artificial intelligence (AI) based conversationality, which is designed to provide information to users and collect information from users. Such systems use an AI core and natural language processing (NLP), deep learning, and language understanding/processing algorithms to accomplish their aims. Conversational AI systems designed specifically for the healthcare domain are lesser in number as compared to other domains.

Electronic medical file systems, also known as telemedicine systems, are known in the art. These telemedicine systems allow medical practitioners to engage in diagnostic activities without being in the same physical location as the patient. For example, some telemedicine systems allow doctors to send and receive diagnostic data, such as x-rays, sonograms, audio data, audiovisual data, graphic data, text data, or other suitable diagnostic data. However, conventional systems lack integration. For example, a physician must access multiple sources of records to assemble a workflow. Further, a physician is likely to encounter inaccuracies and/or gaps in information.

Embodiments of the invention include a system for using automated completion of healthcare processes. The system can use artificial intelligence (AI) systems for automating healthcare workflows, particularly in patient engagement and revenue cycle management (RCM). In aspects, a healthcare professional or administrator can provide oral instruction using natural language. The system can use machine learning and recursive AI reasoning to interpret the instructions. It can extract insights from structured and unstructured data and autonomously execute tasks within electronic medical records (EMR) and insurance systems. As described in the examples herein, the system enables real-time patient interactions, adaptive workflow modifications, automated insurance verification, and discrepancy resolution, enhancing efficiency and accuracy in healthcare operations.

### AI Agent for Conversation Between a User and Healthcare Professional

A practical problem that is often encountered in conversational systems used for TeleHealth consults and in other health IT applications is bringing complete resolution to the user's questions. In the absence of a resolution, users abandon their session leading to undesired outcomes.

The present invention solves this problem by providing accurate and complete information to the user by automatically forwarding certain scenarios to human experts, encountered in TeleHealth and other health applications. This system/method enables the continuity of conversation between a user, conversational system, and health experts, for scenarios when the conversational system is unable to provide a resolution to the users medical conversations. The invention includes methods to intelligently understand the users input, determine if accurate responses are available, and in the absence of which, automatically transfer the conversation to health experts (i.e., a human agents). A human agent can be connected in different scenarios, such as, when a user asks a complex question, when questions indicate medical severity/complexity, when answers are unavailable, or when a question should be specifically handled by a health expert (e.g., related to urgency or a potential crisis).

As medical conversations contain a plethora of medical (i.e., technical) terms, the meaning of input must be carefully understood, as even a slight change in the sequence of words may change the meaning of the input, thus leading to an unresolved scenario.

In the system/method described herein, a Natural Language Processing (NLP) process starts by analyzing the users' inputs by identifying the sentiment and underlying semantics. Such inputs are then validated for accuracy and a severity/complexity threshold score is generated. If the threshold score is above an arbitrary value, then a database match is executed. The database used is a medical scenario database which will help determine further processing of the user's input.

This medical scenario database can include clusters of medical scenarios that should be handled by a health expert. If the user's input is mapped to a cluster with a pre-defined level of confidence that indicates the need for health expert intervention, then the conversation is automatically transferred to the next available health expert. The user continues their conversation with the health expert, which when terminated is redirected back to the automated system.

Additionally, the health expert can also restore the conversation between the user and the system. Multiple methods are used to build the medical scenario database cluster by analyzing a plurality of medical information from a variety of sources. By applying techniques such as medical text analytics, ontologies, etc., medical topics can be identified (e.g., as severity, complexity, certain diagnosis types, treatment options, immediate support, mental health issues, etc.) that require health expert (i.e., human) intervention. Methods of retrieving medical information from health reports using AI are known in the art (see, e.g., U.S. Pat. No. 10,754,882B2). The methods can convert data from medical documents into a knowledge graph using a machine-assisted approach. This approach provides the functionality to ingest and analyze any medical document for downstream processing such as creating this medical scenario database.

In embodiments, the system/methods us an AI-driven agentic methodology that autonomously engages users to complete healthcare-related interactions. The AI agent attempts to resolve queries and process tasks through iterative reasoning, leveraging a medical scenario classifier to map user input to predefined medical or financial scenarios. The agent dynamically references a continuously updated medical scenario database, which integrates medical literature, ontologies, and knowledge bases, to refine its responses and decision-making.

If the AI agent encounters a scenario where resolution remains incomplete after recursive attempts—such as multiple failed clarification loops, confidence score thresholds not being met, or the detection of complex, ambiguous input—the system escalates the conversation to a human expert. This ensures that only cases requiring human expertise are transferred, optimizing efficiency while maintaining accuracy and compliance in patient engagement and revenue cycle management (RCM) processes.

In embodiments, the system enables automation of patient engagement workflows and revenue cycle operations, including tasks such as medical billing, claims reconciliation, patient inquiries, financial assistance assessment, and eligibility verification. When necessary, a human expert can retrieve and restore an AI-facilitated interaction to provide oversight or clarification. By integrating AI-driven automation with human decision-making checkpoints, this system improves process efficiency, reduces administrative overhead, and enhances accuracy in healthcare operations.

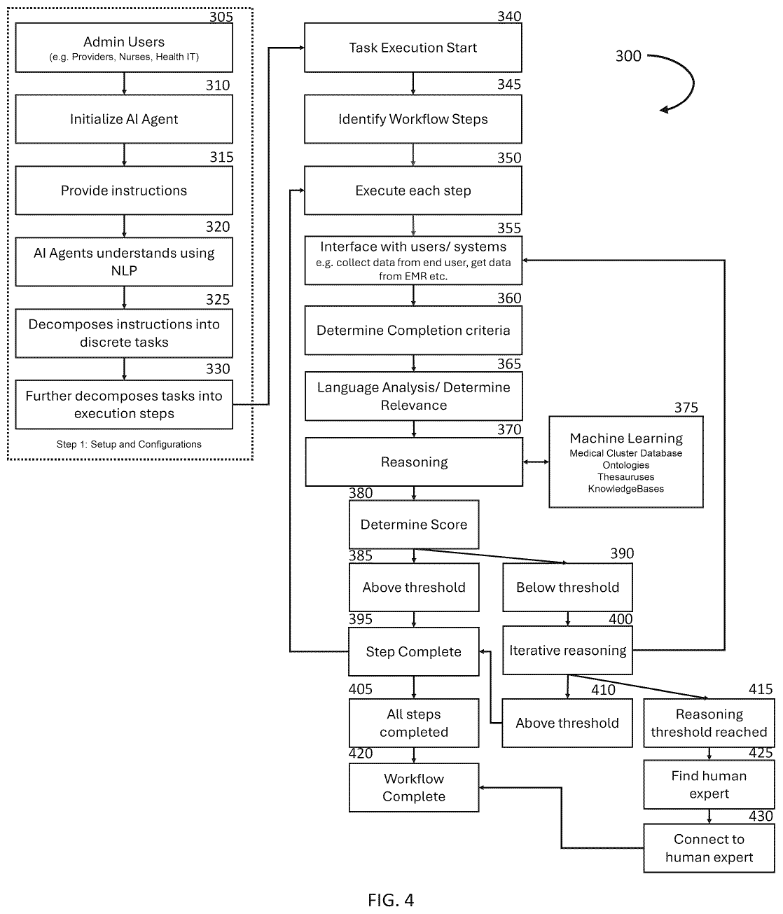

FIG. 4 is a flowchart 300 of a method of creating healthcare process using the system described herein. The first series of steps involves set up and configuration. The user 305 can be a healthcare provider (e.g., doctor, nurse, physician assistant or IT professional) who initiates contact with the system. The provider can do this using normal speech (i.e., natural language content). Next, the system initializes the AI agent 310. The healthcare provider provides verbal instructions 315. The AI agent understands/interprets the instructions using a natural language processor (NLP) 320. Thereafter, instructions can be decomposed into discrete tasks 325 and further decomposed into execution steps 330.

The next series of steps begins with task execution 340. The system identifies workflow steps 345. Next, the system can execute each step 350. An interface can allow for additional information or data collection 355. The system can determine completion criteria 360. Language can be analyzed to determine relevance 365 followed by a reasoning step 370 that can employ machine learning 375.

Based on these steps, the system can determine a score 380. If the score is above a threshold 385, the system can determine that a step is complete 395. Thereafter, the system can continue to the next step 350. Alternatively, if all steps are completed 405, the system can determine if the workflow is complete 420 and a human expert can be connected 430.

If the score is below a threshold 390, the system can apply iterative reasoning 400. Based on this, If the score is above a threshold, the system can conclude that the step is complete 395. Alternatively, if a reasoning threshold is reached 415, the system can find a human expert 425 and connect with the human expert.

In summary, the invention enables automated conversation continuation between a user, system, and health experts. This leads to a complete resolution of users' medical questions increasing user! patient satisfaction.

### Methods of Use

The machine learning software of the invention can be hosted on a cloud server or any other server or server-like infrastructure known in the art.

The machine learning software of the present invention is compatible with any operating system, including but not limited to: Mac OS, Microsoft Windows, Linux, Arthur, ARX, MOS, RISC IX, RISC OS, Fire OS, AmigaOS, Amiga Unix, AMSDOS, Contiki, CP/M 2.2, CP/M Plus, SymbOS, IBM, IBM AIX, Newton OS, iPadOS, watchOS, tvOS, bridgeOS, visionOS, XTS-400, BeOS, BelA, Unix, MINI-UNIX, PWB/UNIX, CB UNIX, BESYS, Inferno, Burroughs MCP, GEOS, AmigaOS, AROS Research Operating System, SCOPE, Chippewa Operating System, MACE, Kronos, NOS, SIPROS, Puffin OS, CTOS, AOS, DG/UX, RDOS, CTOS, Deos, HeartOS, CP/M, Personal CP/M, CP/M Plus, CP/M-68K, CP/M-8000, CP/M-86 Plus, Personal CP/M-86, MP/M, MP/M II, FlexOS, Novell, or any other known operating system in the art.

The machine learning software of the invention is also compatible with myriad graphical user interfaces. Compatible graphical user interfaces include those hosted on desktop computer monitors, laptop computer monitors, smartphone screens, smartwatch screens, television monitors, projection screens, multiplexed screens, LCD displays and other interfaces known in the art.

The machine learning software of the invention can automatically import and compile medical data and medical research from myriad sources. The myriad sources of medical data and medical research include, but are not limited to: medical journals, medical articles, data inputted by professionals in medical fields, medical text books, scientific findings, scientific discoveries, and any and all medical and scientific information available on the Internet that is verified by trusted and reliable sources.

The invention may be described in the general context of computer-executable instructions, such as program modules, being executed by a computer. Generally, program modules include routines, programs, objects, components, data structures, etc., that perform particular tasks or implement particular abstract data types. The computer program and data may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed hard disk), an optical memory device (e.g., a CD-ROM or DVD), a PC card (e.g., PCMCIA card), or other memory device. The computer program and data may be fixed in any form in a signal that is transmittable to a computer using any of various communication technologies, including, but in no way limited to, analog technologies, digital technologies, optical technologies, wireless technologies, networking technologies, and internetworking technologies. The computer program and data may be distributed in any form as a removable storage medium with accompanying printed or electronic documentation (e.g., shrink wrapped software or a magnetic tape), preloaded with a computer system (e.g., on system ROM or fixed disk), or distributed from a server or electronic bulletin board over the communication system (e.g., the Internet or World Wide Web.) It is appreciated that any of the software components of the present invention may, if desired, be implemented in ROM (read-only memory) form. The software components may, generally, be implemented in hardware, if desired, using conventional techniques.

The invention may also be practiced in distributed computing environments where tasks are performed by remote processing devices that are linked through a communications network. In a distributed computing environment, program modules may be located in both local and remote computer storage media including memory storage devices. Practitioners of ordinary skill will recognize that the invention may be executed on one or more computer processors that are linked using a data network, including, for example, the Internet. In another embodiment, different steps of the process can be executed by one or more computers and storage devices geographically separated but connected by a data network in a manner so that they operate together to execute the process steps. In one embodiment, a user's computer can run an application that causes the user's computer to transmit a stream of one or more data packets across a data network to a second computer, referred to here as a server. The server, in turn, may be connected to one or more mass data storage devices where the database is stored. The server can execute a program that receives the transmitted packet and interpret the transmitted data packets in order to extract database query information. The server can then execute the remaining steps of the invention by means of accessing the mass storage devices to derive the desired result of the query. Alternatively, the server can transmit the query information to another computer that is connected to the mass storage devices, and that computer can execute the invention to derive the desired result. The result can then be transmitted back to the user's computer by means of another stream of one or more data packets appropriately addressed to the user's computer. In one embodiment, the relational database may be housed in one or more operatively connected servers operatively connected to computer memory, for example, disk drives. In yet another embodiment, the initialization of the relational database may be prepared on the set of servers and the interaction with the user's computer occur at a different place in the overall process.

Further illustration of the present invention is shown in the working examples produced below.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Use Case of the AI Agent in Healthcare Workflow Automation

The AI agent receives task input in natural language from a healthcare provider and interprets it using natural language processing (NLP). It then decomposes the task into an adaptive, multi-step workflow, leveraging both structured and unstructured data sources to execute actions within the electronic medical record (EMR), insurance systems, and other healthcare platforms. The AI agent dynamically interacts with patients via text or voice-based conversations, allowing it to modify workflows in real-time based on user responses, identify potential medical risks or severe conditions, and adjust the next steps accordingly. Additionally, the system can validate insurance eligibility in real-time for one or multiple payers and automatically identify commonly executed procedures and tests, triggering prior authorization requests when required. This ensures that necessary approvals are obtained seamlessly before scheduling services, reducing administrative delays and claim denials.

In this example, a task is given to AI Agent (User Input):
“Find all patients in the due date for an annual checkup, including those who had a recent encounter a year ago. Update or gather their demographics. Have them fill out the general health questionnaire. If male and 40 years old, include a colon cancer screening form. If they have a history of heart conditions, include a heart health questionnaire. Perform real-time insurance eligibility verification for all applicable plans. Identify commonly required procedures or tests and obtain prior authorization if needed and in real-time. Update insurance details, schedule them for an appointment, and notify the clinic admin. Prepare the patient's chart for my review. If any discrepancies arise, route them to a nurse.”

### AI Agent Workflow Execution

In this example, the system executes the following workflow:
1. Identify Patients Due for Checkup (Structured+Unstructured Data Analysis)
   a) Query the EMR for structured data such as last visit dates and upcoming preventive care needs.
   b) Process unstructured data (e.g., physician notes, scanned documents, historical PDFs) using AI-driven text analysis to extract relevant indicators.
   c) Cross-check with clinical guidelines to confirm eligibility for preventive care (e.g., annual checkups, screenings).
2. Perform Real-Time Insurance Eligibility Verification & Prior Authorizations
   a) Query one or multiple insurance providers in real time to check coverage status for annual checkups, lab tests, and screenings.
   b) Detect discrepancies in coverage (e.g., expired policies, pending authorizations) and flag them for resolution.
   c) Provide alternative payment options or financial assistance recommendations if necessary.
   d) Perform real-time prior-authorizations for recommended procedures & test.
3. Update or Gather Patient Demographics
   a) Extract existing patient demographic data from the EMR.
   b) If missing or outdated, request updates directly from the patient via chatbot, voicebot, or patient portal.
   c) Confirm details such as contact information, emergency contacts, and preferred language.
4. Administer Adaptive Health Questionnaires
   a) Send the general health questionnaire to all identified patients.
   b) Personalize forms dynamically:
      If male and 40 years old, attach the colon cancer screening form.
      If a history of heart conditions is detected, include the heart health questionnaire.
   c) AI monitors patient responses in real-time to detect potential red flags (e.g., reporting chest pain→immediate escalation to a nurse).
5. Conversational AI Interaction & Dynamic Workflow Adjustments
If a patient provides unexpected or contradictory answers, the AI dynamically modifies the workflow.
Example: If a patient mentions recent severe symptoms while filling out a form, the AI:
   Adjust the next steps (e.g., instead of scheduling a routine visit, it may recommend an urgent evaluation).
   Notifies a healthcare professional if symptoms indicate a potential emergency.
6. Schedule Appointments Efficiently.
   Check physician availability and automatically match patients to the most suitable time slot.
   Suggest telehealth vs. in-person visits based on patient preference and medical necessity.
7. Notify Clinic Admin & Prepare Physician Charts
   a) Generate a structured summary of patient updates, completed forms, and flagged issues.
   b) Prepare and organize charts for physician reviews, including:
      Recent medical history, questionnaire responses, and flagged concerns.
      Verified insurance details and coverage recommendations.
8. Discrepancy Handling & Escalation
   a) Identify missing or conflicting data (e.g., patient self-reports smoking cessation, but EMR shows active tobacco use).
   b) AI attempts recursive resolution (e.g., requesting clarification from the patient or reconciling data sources).
   c) If unresolved after iterative attempts, the AI escalates the case to a nurse or human administrator for intervention.

### Example 2

### Use AI Agent in Revenue Cycle Management

In this example, a task is given to AI Agent (User Input):
“Process all upcoming scheduled appointments for the next week. Perform comprehensive insurance verification for each patient to confirm active coverage and benefits. Determine which procedures require prior authorization and initiate those requests immediately. Verify patient responsibility amounts and generate cost estimates. For surgical procedures, review clinical documentation and suggest appropriate CPT and ICD-10 codes. Identify any missing demographic information and update patient records. Flag high-risk cases where insurance might deny coverage. Send personalized notifications to patients about their estimated out-of-pocket costs. Create a daily summary report of financial clearance status for all appointments. Alert the scheduling team about any cases requiring immediate attention due to insurance issues.”

### AI Agent Workflow Execution

In this example, the system executes the following workflow:
1. Process and Organize Upcoming Appointments
   a) The AI agent analyzes the schedule to identify all appointments requiring financial clearance.
   b) Applies contextual understanding to prioritize appointments based on:
      Procedure complexity and cost
      Insurance payer requirements
      Historical patterns of payment issues
      Lead time needed for authorization
      The agent recognizes nuanced differences between appointment types requiring different verification workflows.
2. Comprehensive Insurance Verification (Adaptive Learning)
   a) The AI intelligently connects with multiple payer platforms and portals simultaneously.
   b) Cognitively extracts and interprets complex benefit information, including:
      Plan status (active/inactive).
      Coverage details for specific services.
      Network status of providers
      Benefit limitations and exclusions
      Remaining deductibles and out-of-pocket maximums
   c) The agent applies learned patterns from thousands of previous verifications to identify potential coverage issues.
   d) Recognizes and flags unusual benefit structures or coverage gaps that might require special attention.
3. Prior Authorization Management (Reasoning and Prediction)
   a) The AI agent reasons through medical necessity criteria for scheduled procedures.
   b) Identifies procedures requiring authorization by analyzing:
      CPT/HCPCS codes
      Payer-specific authorization requirements
      Patient diagnosis and clinical history
      The agent predicts authorization outcomes based on historical patterns and current clinical documentation.
   c) Autonomously initiates authorization requests by:
      Gathering relevant clinical documentation from the EMR
      Completing payer-specific authorization forms
      Submitting requests through appropriate channels (portals, fax, phone)
   d) Continuously monitors authorization status and escalates stalled requests.
4. Patient Financial Responsibility Calculation (Mathematical Modeling)
   a) The AI applies complex benefit calculations to determine patient responsibility.
   b) Cognitively processes multiple variables simultaneously:
      Contract-specific allowed amounts
      Multiple insurance coordination rules
      Service-specific benefit structures
      Accumulated deductibles and out-of-pocket maximums
   c) The agent handles exception scenarios such as:
      Services with percentage-based coinsurance vs. fixed copayments
      Items excluded from coverage but part of a bundled service
      Services with quantity or frequency limitations
5. Medical Coding Analysis and Suggestion (Knowledge Integration)
   a) The AI comprehends clinical documentation to identify relevant diagnoses and procedures.
   b) Applies sophisticated understanding of coding guidelines and conventions.
   c) The agent cognitively maps clinical terminology to appropriate ICD-10 and CPT codes.
   d) Recommends optimal code combinations that accurately represent:
      Medical necessity for the procedure
      Complexity of the service provided
      Relevant comorbidities and complications
      The agent recognizes potential coding issues such as:
      Missing documentation elements needed for specific codes
      Potential code pairs requiring modifiers
      Diagnosis codes that may not support medical necessity
6. Patient Communication and Engagement (Emotional Intelligence)
   a) The AI tailors financial communications based on patient preferences and history.
   b) Generates personalized cost estimates that clearly explain:
      Expected insurance coverage
      Estimated out-of-pocket responsibility
      Payment options available
      The agent adapts communication style and detail level based on:
      Patient's previous questions or concerns about billing
      Complexity of the financial information
      Patient's demonstrated health literacy in past interactions
7. Risk Assessment and Issue Identification (Predictive Analytics)
   a) The AI cognitively identifies high-risk scenarios that may lead to payment delays.
   b) Applies predictive modeling to flag cases likely to require intervention:
      Procedures commonly denied by specific payers
      Authorization requirements likely to be questioned
      Benefit limitations that may impact coverage
      The agent autonomously determines appropriate risk mitigation strategies.
      Escalates critical issues to human staff with clear context and suggested actions.
8. Reporting and Analytics (Pattern Recognition)
   a) The AI synthesizes complex data into actionable financial clearance reports.
      Cognitively organizes information by relevance and urgency for different stakeholders.
   b) The agent identifies emerging patterns in insurance verification challenges.
   c) Generates insights that can improve future revenue cycle processes, such as:
      Payers with increasing authorization requirements
      Services frequently requiring additional documentation
      Emerging reimbursement trends by payer or service line

### Example 3

### Use AI Agent in Healthcare Workflow Automation

In this example, a task is given to AI Agent (User Input):
“Find all patients in the due date for an annual checkup, including those who had a recent encounter a year ago. Update or gather their demographics. Have them fill out the general health questionnaire. If male and 40 years old, include a colon cancer screening form. If they have a history of heart conditions, include a heart health questionnaire. Perform real-time insurance eligibility verification for all applicable plans. Identify commonly required procedures or tests and obtain prior authorization if needed and in real-time. Update insurance details, schedule them for an appointment, and notify the clinic admin. Prepare the patient's chart for my review. If any discrepancies arise, route them to a nurse.”

### AI Agent Workflow Execution

1. Identify Patients Due for Checkup (Structured+Unstructured Data Analysis)
   a) Query the EMR for structured data such as last visit dates and upcoming preventive care needs.
   b) Process unstructured data (e.g., physician notes, scanned documents, historical PDFs) using AI-driven text analysis to extract relevant indicators.
   c) Cross-check with clinical guidelines to confirm eligibility for preventive care (e.g., annual checkups, screenings).
2. Perform Real-Time Insurance Eligibility Verification & Prior Authorizations
   a) Query one or multiple insurance providers in real time to check coverage status for annual checkups, lab tests, and screenings.
   b) Detect discrepancies in coverage (e.g., expired policies, pending authorizations) and flag them for resolution.
   c) Provide alternative payment options or financial assistance recommendations if necessary.
   d) Perform real-time prior-authorizations for recommended procedures & test.
3. Update or Gather Patient Demographics
   a) Extract existing patient demographic data from the EMR.
   b) If missing or outdated, request updates directly from the patient via chatbot, voicebot, or patient portal.
   c) Confirm details such as contact information, emergency contacts, and preferred language.
4. Administer Adaptive Health Questionnaires
   a) Send the general health questionnaire to all identified patients.
   b) Personalize forms dynamically:
      If male and 40 years old, attach the colon cancer screening form.
      If a history of heart conditions is detected, include the heart health questionnaire.
   c) AI monitors patient responses in real-time to detect potential red flags (e.g., reporting chest pain→immediate escalation to a nurse).
5. Conversational AI Interaction & Dynamic Workflow Adjustments
   a) If a patient provides unexpected or contradictory answers, the AI dynamically modifies the workflow.
   b) Example: If a patient mentions recent severe symptoms while filling out a form, the AI:
      Adjust the next steps (e.g., instead of scheduling a routine visit, it may recommend an urgent evaluation).
      Notifies a healthcare professional if symptoms indicate a potential emergency.
6. Schedule Appointments Efficiently
   a) Check physician availability and automatically match patients to the most suitable time slot.
   b) Suggest telehealth vs. in-person visits based on patient preference and medical necessity.
7. Notify Clinic Admin & Prepare Physician Charts
   a) Generate a structured summary of patient updates, completed forms, and flagged issues.
   b) Prepare and organize charts for physician reviews, including:
      Recent medical history, questionnaire responses, and flagged concerns.
      Verified insurance details and coverage recommendations.
8. Discrepancy Handling & Escalation
   a) Identify missing or conflicting data (e.g., patient self-reports smoking cessation, but EMR shows active tobacco use).
   b) AI attempts recursive resolution (e.g., requesting clarification from the patient or reconciling data sources).
   c) If unresolved after iterative attempts, the AI escalates the case to a nurse or human administrator for intervention.

### Example 4

Male Patient with Chronic Migraine

Common reasons that one contacts and/or visits a doctor include:
a) Skin Disorders
b) Joint Pain and Osteoarthritis
c) Back Problems
d) Cholesterol Problems
e) Upper Respiratory Problems (not including asthma)
f) Anxiety, Bipolar Disorder and Depression
g) Chronic Neurology Disorders
h) High Blood Pressure
i) Headaches and Migraines
j) Diabetes

In this example, a 54-year-old male patient reports to his doctor that he is experiencing chronic and acute frontal temporal pain. He contacts the agentic AI agent with a series of questions.
Why do I have chronic migraines?
Is there any way to reduce the severity of my symptoms?
What is the likelihood that it is caused by stress?
What is the likelihood that it is caused by something more serious (e.g., stroke)?
Do I need treatment or will aspirin help?
Should I be tested for stroke by getting a CT scan?
What changes should I make to my lifestyle/diet?
Is there any procedure that could help alleviate my headaches?

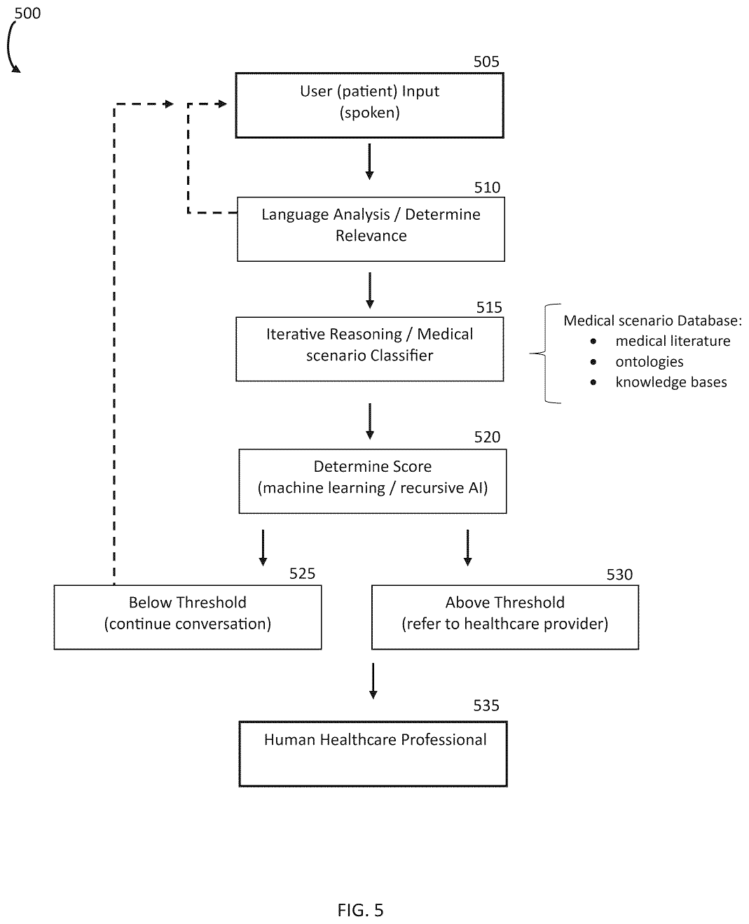

The agentic AI agent system responds to each question using the method shown in FIG. 5. The patient initiates contact with the system using an App or smart phone 505. The system uses language analysis to categorize statements and determine relevance 510. Iterative reasoning is implemented using a medical database 515. Based on these steps, the system can determine a score for each statement/query 520. If the score is below a threshold, the system can continue the conversation 525. If the score is above a threshold, the system can refer the patient to a healthcare provider 530. The provider can be brought into the conversation 535 (e.g., via direct phone call). If a healthcare provider is unavailable, a message can be relayed to the provider. Alternatively, the patient can be advised to visit a clinic or hospital. In this example, the statements/queries presented by the patient remain below the threshold.

The patient is advised that mild to severe pain in the forehead or temples can be caused by stress, infections, dehydration, poor posture or eye strain. Based on the signs, symptoms, patient health and additional biomedical information, the system determines that the patient has a very low likelihood of stroke or other urgent condition. The patient is advised to take an OTC pain medication (e.g., acetaminophen, aspirin or ibuprofen). Additionally, the agent recommends getting adequate rest, managing stress and staying hydrated. The patient is advised to seek further medical attention if the headaches are persistent, worsening or very severe.

### Example 5

Female Patient with Kidney Stone

A 71-year-old female patient complains of pain in her lower abdomen. The patient explains that she has had kidney stones in the past. She presents several questions:
Do I have a kidney stone or is there another reason for my symptoms?
What type of kidney stone do I have?
What size is my kidney stone?
Where is my kidney stone located?
How many kidney stones do I have?
Do I need treatment or will I be able to pass the kidney stone?
Should I be tested for kidney disease?
What changes should I make to my diet?
What type of procedure should I have to get rid of the stones?

As in the example above, the patient initiates contact with the system using an App or smart phone 405. The system determines a score for each statement/query 420. Because of the severity of pain and complexity of questions, the system determines that such is above a threshold. A healthcare provider is contacted 435 via direct phone. The healthcare provider doctor recommends a urologist who can perform a CT and treat the patient for kidney stones if needed.

### Example 6

### Workflow for Contacting Category of Patients

In this example, a healthcare provider wishes to contact a specific group of patients. A new medication is available to treat hyperlipidemia. The provider tasks the system to create a list of patients who have received a serum lipid profile with the following criteria:
Age 45 or older
Total cholesterol ≥200 mg/dl
LDL cholesterol ≥130 mg/dl
Triglycerides ≥150 mg/dl (or ≥200 mg/dl if on medication)
HDL cholesterol <40 mg/dl

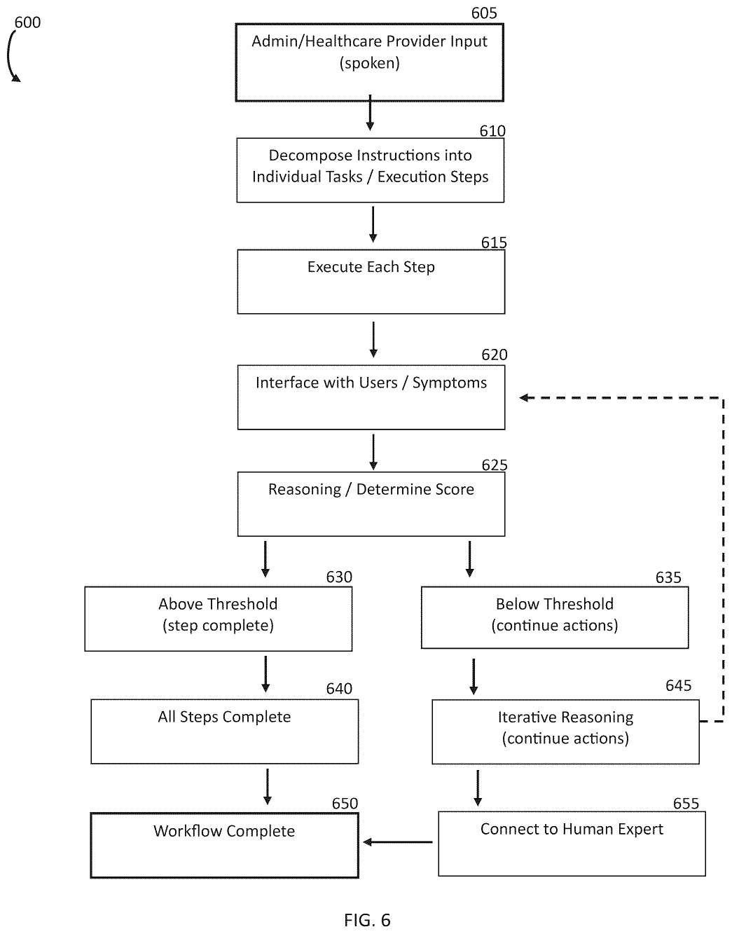

The provider describes this criteria to the agentic AI agent system using the method shown in FIG. 6. The provider uses an App or smart phone 605. As above, the system uses language analysis to categorize statements and determine relevance. Next, the instructions are used to create a series of tasks or steps 610. In this example, each patient file is reviewed for age and lipid levels 615. Iterative reasoning is implemented using a medical database. Additional information can be obtained from an interface (e.g., input from medical staff related to insurance) 620. Based on these steps, the system can determine a score for each step 625. If the score is above a threshold, the system can conclude that a step is complete 630. Thereafter, the system can continue executing each step until all are complete 640 and the workflow is complete 650.

If the score is below a threshold, the system can continue actions 635. For example, if there is uncertainty in a patient's file, the system can initiate input via the interface 620 and continue the process. If the uncertainty is not resolved, the system can connect with a human expert 655.

The healthcare provider receives the list of patients who satisfy the above criteria. The system can also be used prioritize patients (e.g., establish a que) so that those who are at highest risk of ailments related to hyperlipidemia are contacted first. Further, the system can initiate contact with each patient to inform each of the treatment and/or schedule a consultation.

### Example 7

### Use of AI Agent in Patient Engagement

In this example, a healthcare provider wishes to contact a specific group of patients. The provider tasks the system with the following instructions:
“Identify patients scheduled for elective surgeries in the next month. Reach out to patients the update their medical history, medications, and allergies-keeping everything accurate and up to date.
Next, they'll get educational content specific to their procedure, including pre-op instructions and post-op care plans. Acquire digital consent by easy review and e-signing, ensuring a smooth approval process. Meanwhile, run real-time insurance checks and handle prior authorizations for the procedure.
To keep everything on track, schedule pre-op consultations, with notifications sent to both patients and surgical teams for seamless coordination. As the surgery date approaches, all necessary patient information-like lab results and imaging-will be compiled into the patient's chart for the surgical team's review.
Finally, post-op follow-up appointments will be arranged, with reminders sent to ensure a smooth recovery and continued care!”

The healthcare provider receives the list of patients who satisfy the above criteria. The system contacts patients with instructions and schedules visits to the office and clinic. Further, the system can initiate contact between a patient and healthcare provider and/or schedule a consultation as needed (e.g., when urgent or at the patient's request).

In aspects, the system and/or method reduces the time to match and/or transfer a patient presenting with a condition (e.g., high blood pressure, etc.) to a healthcare professional or specialist.

In aspects, the methods provide a parallel process to a traditional workflow (e.g., standard radiology workflow), which can confer the benefit of reducing the time to determine a treatment option while having the outcome of the traditional workflow as a backup in the case that an inconclusive or inaccurate determination (e.g., false negative, false positive, etc.) results from the method.

In aspects, the methods are configured to have a high sensitivity (e.g., greater than 90%), which functions to detect a high number of true positive cases with a disease/ailment and help these patients reach treatment faster.

In aspects, the methods confer the benefit of reorganizing a queue of patients, wherein patients having a certain condition are detected early and prioritized (e.g., moved to the front of the queue).

In some aspects, the method confers the benefit of determining actionable analytics to optimize a workflow, such as an emergency room triage workflow.

In aspects, the method and/or system confers the benefit of identifying a patient suited for a particular clinical trial. For example, an assessment of the patient can be used to check for the patient's eligibility for a clinical trial, and can optionally initiate any suitable triggers (e.g., notifying the patient, sending the patient information regarding the clinical trial, notifying a principal investigator [PI] for the clinical trial, etc.). This can increase in the number of individuals recommended for a clinical trial, provide a better match between the individuals recommended for a clinical trial and the objectives of the clinical trial, reduce the number of individuals who are recommended for a clinical trial but do not qualify, etc.

In aspects, the systems/methods use natural language generation (NLG) to summarize data in natural language text, where input may be values in tables, lists of keywords, key-value pairs, knowledge graph entries, etc. The system does not require explicit supervision (e.g., training data can be automatically generated, etc.), and may utilize available natural language processing (NLP) systems as a source for generating training data. In aspects, the system can detect part-of-speech (POS) tags of keywords using a context oblivious approach.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter. Further, the use of the terms “include,” “includes” and “including” means include, includes and or including as well as include, includes and including, but not limited to.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

Although embodiments of the current disclosure have been described comprehensively in considerable detail to cover the possible aspects, those skilled in the art would recognize that other versions of the disclosure are also possible.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

What is claimed is:

1. A method for machine-assisted healthcare workflow generation, the method comprising steps of:
a) interpreting user instructions and extracting content using a natural language processor (NLP),
b) decomposing the extracted intent into logical workflows tasks with a workflow generation engine,
c) decomposing tasks into multi-step executable actions referencing healthcare systems, and
d) mapping the tasks to predefined medical, administrative, and financial workflows, using a medical scenario classifier.

2. The method of claim 1, further comprising a step of: e) resolving one or more of the logical workflow tasks.

3. The method of claim 1, further comprising a step of: e) resolving discrepancies and routing conversations to a healthcare expert using a resolution manager.

4. The method of claim 1, further comprises a step of building a medical database cluster to store medical scenarios that require input from a health expert,
wherein the medical database cluster comprises data on signs/symptoms of medical conditions, data on severity of medical conditions, lab test results and social determinants of health and drugs and side-effects of drugs.

5. The method of claim 1, wherein a user submits user instructions by spoken natural language.

6. The method of claim 1, wherein the step of interpreting user instructions and extracting content further comprises language analysis of the user's input using one or more of sentiment analysis and contextual identification of medical terminologies.

7. The method of claim 1, further comprising a step of retrieving and processing structured and unstructured medical data to optimize task execution.

8. The method of claim 1, further comprising a step of verifying user credentials, insurance records, and/or patient eligibility for medical treatment or a medical procedure.

9. A system for machine-assisted healthcare workflow generation, the system comprised of:
a) a natural language processing (NLP) module configured to interpret user instructions and extract intent,
b) a workflow generation engine configured to decompose the extracted intent into logical workflows tasks with recursion, and decompose tasks into multi-step executable actions referencing healthcare systems,
c) a medical scenario classifier configured to map tasks to predefined medical, administrative, and financial workflows, and
d) a resolution manager that can resolve discrepancies and route conversations to an expert when deemed necessary.

10. The system of claim 9, further comprising a medical database cluster,
wherein the medical database cluster comprises data on signs/symptoms of medical conditions, data on severity of medical conditions, lab test results and social determinants of health and drugs and side-effects of drugs.

11. The system of claim 9, further comprising a database of structured and unstructured medical data.

12. The system of claim 9, further comprising a database for verifying user credentials, insurance records, and/or eligibility for medical treatment or a medical procedure.

13. A method for machine-assisted automated continuation of conversations between a user, software system and healthcare professional, the method comprising steps of:
a) submitting user instructions to an agentic AI system,
b) validating if the user's inputs are medically relevant,
c) annotating the user's input for one or more of context, semantics, ontologies, medical terms and medical phrases,
d) mapping the inputs to the medical database cluster,
e) establishing a scenario assessment score based on combining information from mapping relevance, medical database cluster and ontologies, and
f) determining a score based on relevance, routing the conversation to a health expert or providing a response to the user and allowing the user to resume communication.

14. The method of claim 13, further comprising a step of building a medical database Taking as input the outcomes from the AI agent that was attempting to resolve the workflow task but determined need for human intervention, followed by

15. The method of claim 13, further comprising a step of building a medical database cluster to store medical scenarios that require input from a health expert, the medical database clusters comprising data on signs/symptoms of medical conditions, data on severity of medical conditions, drugs and side-effects of drugs.

16. The method of claim 13, further comprising a step of language analysis of the user's input using one or more of sentiment analysis and contextual identification of medical terminologies.

17. The method of claim 13, further comprising a step of sending context of the conversation to the health expert.

18. The method of claim 13, further comprising a step of storing anonymized conversations that from the user and health expert for model training.

19. The method of claim 13, wherein the user submits the user instructions by spoken natural language.

20. The method of claim 19, further comprising the steps of: Implementing a text-voice conversational system that is managed by a command, allowing the user to enter queries in natural language, and validating the user's queries for medical accuracy.
